(19) United States
(12) Patent Application Publication
(10) Pub. No.: US 2017/0065659 A1
(43) Pub. Date: Mar. 9, 2017

(54) COMPOSITION FOR USE IN TREATING AND/OR PREVENTING HUMAN CONDITION, DISORDER OR DISEASES

(71) Applicant: Birgit Christine SKORPEN, Berg (DE)

(72) Inventor: Birgit Christine SKORPEN, Berg (DE)

(21) Appl. No.: 15/256,852

(22) Filed: Sep. 6, 2016

(30) Foreign Application Priority Data
Sep. 7, 2015 (DE) 10 2015 011 712.2

(51) Int. Cl. A61K 36/899; A61K 45/06; A61K 31/047; A61K 36/73; A61K 33/32; A61K 33/24

(52) U.S. Cl. CPC A61K 36/899; A61K 33/32; A61K 33/24; A61K 31/047; A61K 36/73; A61K 45/06

(57) ABSTRACT

Composition comprising a metal and/or a semimetal, a sodium salt, a tranquilizer, a halogen, a carotenoid, grain, and a neutral gel for example for use in treating and/or preventing a condition, disorder or disease such as pain, metabolic diseases, fatigue, absence of appetite, cramps, immune diseases, inflammatory disease, cancer, neurological and/or psychiatric diseases.

The invention refers to a composition comprising natural products for use in treating and/or preventing a condition, disorder or diseases for example selected from the group consisting of pain, metabolic diseases, fatigue, absence of appetite, cramps, immune diseases, inflammatory disease, cancer, neurological and/or psychiatric diseases.

Numerous medicaments are available on the market having a very specific treatment focus and severe side effects which are difficult for example for use in treating children, elderly people or patients in a late phase of a severe (chronic) disease. A lot of the active agents in the medicaments, which are meanwhile chemically synthesized, were originally isolated from nature, particularly from plants. In naturopathic treatment still use is made of these active agents in plants which may be available in a lower concentration than after a technical synthesis but remain in their natural environment in combination with other plant compounds.

The problem underlying the present invention is the improvement of a composition for use in treating and/or preventing of a broad range of conditions, disorders or diseases having low side effects.

A solution to this problem is provided by the composition according to claim 1 comprising a metal and/or a semimetal, a sodium salt, a tranquilizer, a halogen, a carotenoid, grain, and a neutral gel.

The composition of the present invention is a medicament after passing through clinical trials and Marketing Authorization proceedings, is a dietary supplement available for example in a health food shop, or used in naturopathic treatment. The combination of the different components provides a broad basis for variation of the composition to adapt it for use in the treatment and/or prevention of a condition, disorder or diseases. The composition of claim 1 comprises or consists of one or more of the different components, i.e., one or more of the metal, semimetal, sodium salt, tranquilizer, halogen, carotenoid and grain. The components of the composition of the present invention can be administered to a subject at a suitable dose.

In the following, the elements of the present invention will be described in more detail. These elements are listed with specific embodiments, however, it should be understood that they may be combined in any manner and in any number to create additional embodiments. The variously described examples and embodiments should not be construed to limit the present invention to only the explicitly described embodiments. This description should be understood to support and encompass embodiments which combine the explicitly described embodiments with any number of the disclosed elements. Furthermore, any permutations and combinations of all described elements in this application should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps. The terms “a” and “an” and “the” and similar reference used in the context of describing the invention (especially in the context of the claims) are to be construed to cover both the singular and the plural, unless otherwise indicated herein or clearly contradicted by the context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”, “for example”), provided herein is intended merely to better illustrate the invention and does not pose a limitation on the scope of the invention otherwise claimed. No language in the specification should be construed as indicating any non-claimed element essential to the practice of the invention.

Different metals and semimetals play an important role in the health of mammalians for example chromium, manganese, silicon, iron, cobalt, copper, zinc, calcium, potassium, magnesium and sodium, optionally in different valences, e.g., in the form of different salts. Manganese for example is an important metal for human health, being absolutely necessary for development, metabolism, and the antioxidant system. The classes of enzymes that have manganese cofactors are very broad, and include oxidoreductases, transferases, hydrolases, lyases, isomerases, ligases, lectins, and integrins. The best-known manganese-containing polypeptides is Mn-containing superoxide dismutase (Mn-SOD). Mn-SOD is the type of SOD present in eukaryotic mitochondria, and also in most bacteria. The human body contains about 12 mg of manganese, which is stored mainly in the bones. The remaining manganese in soft tissue is mostly concentrated in the liver and kidneys. In the human brain, the manganese is bound to manganese metalloproteinase, most notably glutamine synthetase in astrocytes. Manganese optionally forms part of the present composition, wherein the composition comprises manganese or any salt thereof in an amount of for example 1 to 100 mg, 5 to 80 mg, 10 to 60 mg, 10 to 50 mg, 15 to 25 or 30 mg.

Another important metal is chromium. Studies suggest that the biologically active form of chromium (III) is an oligopeptide called Low-molecular-weight chromium-binding substance (LMWCr), which might play a role in the insulin signaling pathway. Moreover, silicon is needed for example for synthesis of elastin and collagen and the aorta contains the highest quantity of elastin and silicon. Chromium II and/or III optionally forms part of the present composition, wherein the composition comprises chromium or any salt thereof in an amount of for example 50 to 500 pg, 100 to 500 pg, 150 to 300 pg, 200 or 250 pg.

A further component of the composition of the invention described herein is iron which is abundant in biology and iron-proteins are found in all living organisms, ranging from the evolutionarily primitive archaea to humans. The color of blood is due to the hemoglobin, an iron-containing protein. As illustrated by hemoglobin, iron is often bound to cofactors, e.g. in hemes. The iron-sulfur clusters are pervasive and include nitrogenase, the enzymes responsible for biological nitrogen fixation. Iron is a necessary trace element found in nearly all living organisms. Iron-containing enzymes and proteins, often containing heme prosthetic groups, participate in many biological oxidations and in transport for examples hemoglobin, myoglobin, and cytochrome P450. These compounds can transport gases, build enzymes, and are used in transferring electrons. Metalloproteins are a group of proteins with metal ion cofactors. Some examples of iron metalloproteins are ferritin and rubredoxin.

Also cobalt is essential to all mammalians. It is a key constituent of cobalamin, also known as vitamin B₁₂. Bacteria in the guts of ruminant animals convert cobalt salts into vitamin B₁₂, a compound which can only be produced by bacteria or archaea. The minimum presence of cobalt in soils therefore markedly improves the health of grazing animals, and an uptake of 0.20 mg/kg a day is recommended for them, as they can obtain vitamin B₁₂ in no other way. Non-ruminant herbivores produce vitamin B₁₂ from bacteria in their colons which again make the vitamin from simple cobalt salts. However the vitamin cannot be absorbed from the colon, and thus non-ruminants must ingest feces to obtain the nutrient Animals that do not follow these methods of getting vitamin B₁₂ from their own gastrointestinal bacteria or that of other animals, must obtain the vitamin pre-made in other animal products in their diet, and they cannot benefit from ingesting simple cobalt salts. In humans, B₁₂ exists with two types of alkyl ligand: methyl and adenosyl. MeB₁₂ promotes methyl (—CH₃) group transfers. The adenosyl version of B12 catalyzes rearrangements in which a hydrogen atom is directly transferred between two adjacent atoms with concomitant exchange of the second substituent, X, which may be a carbon atom with substituents, an oxygen atom of an alcohol, or an amine Methylmalonyl coenzyme A mutase (MUT) converts MM1-CoA to Su-CoA, an important step in the extraction of energy from proteins and fats. Although far less common than other metalloproteins (e.g. those of zinc and iron), cobaltoproteins are known aside from B₁₂. These proteins include methionine aminopeptidase 2 an enzyme that occurs in humans and other mammals which does not use the corrin ring of B₁₂, but binds cobalt directly. Another non-corrin cobalt enzyme is nitrile hydratase, an enzyme in bacteria that are able to metabolize nitriles.

A composition of the present invention may comprise additionally a vitamin such as A1, A2, B1, B2, B3, B5, B6, B7, B8, B9, B11, B15, B17, C, D2, D3, E, F, H (biotin), K1, K2, M, inositol, choline, citrine, and/or cabagine.

Another component of the composition is copper, wherein copper proteins have diverse roles in biological electron transport and oxygen transportation, processes that exploit the easy interconversion of Cu(I) and Cu(II). In cytochrome C oxidase, which is required for aerobic respiration, copper and iron cooperate in the reduction of oxygen. Copper is also found in many superoxide dismutases, proteins that catalyze the decomposition of superoxides by converting it (by disproportionation) to oxygen and hydrogen peroxide: 2 HO₂→H₂O₂+O₂.

Further, zinc is an essential trace element for mammalians, for plants and for microorganisms. Zinc is found in numerous enzymes of all enzyme classes, it serves as structural ions in transcription factors and is stored and transferred in metallothioneins. In proteins, Zn ions are often coordinated to the amino acid side chains of aspartic acid, glutamic acid, cysteine and histidine. There are 2-4 grams of zinc distributed throughout the human body. Most zinc is in the brain, muscle, bones, kidney, and liver, with the highest concentrations in the prostate and parts of the eye. In humans, zinc plays ubiquitous biological roles. It interacts with a wide range of organic ligands, and has roles in the metabolism of RNA and DNA, signal transduction, and gene expression and regulates apoptosis. In the brain, zinc is stored for example in specific synaptic vesicles by glutaminergic neurons and can modulate brain excitability. It plays a key role in synaptic plasticity and so in learning

Calcium is the most abundant inorganic element in the higher animals and is located principally in the bones and teeth as apatite, a calcium phosphate mineral. Blood is also a huge reservoir of calcium in mammals. Calcium is distributed throughout all tissues where it has special roles in controlling nerve impulse transmission, muscle action, blood clotting and cell permeability. Calcium deficiency is exhibited by the onset of rickets, failure of the blood-clotting mechanism, nervous disorder and convulsive muscular contractions. Potassium can also be found in the animals' cellular liquids in the form of monopositive ion and has an extremely important role in several biological reactions and vital manifestations, such as transmission of nervous pulses to produce muscular contraction.

The adult human daily nutritional requirement of magnesium, which is affected by various factors including gender, weight, and size, is 300-400 mg/day. Disturbances of the magnesium concentration causes muscle spasms, and has been associated with cardiovascular disease, diabetes, high blood pressure, anxiety disorders, and cerebral infarction. Sodium is likewise an essential biological element to the more advanced animals; the ratio sodium/potassium concentrations in intercellular and extracellular fluids is responsible for the transport ions through the cellular membranes, the regulation of the osmotic pressure inside the cell, the transmission of nervous pulses and other electrophysiological functions. A sodium salt according to the present invention may be selected from the group consisting of NaCl, Na₂SO₄, Na₂CO₃, NaBr, Na₂CrO₄, NaHSO₄, NaOH, NaMnO₄, Na₃PO₄, Na₂HPO₄ and NaH₂PO₄. Na₂SO₄.10 H₂O (Glauber's salt) is used in medicine for example as a mild laxative.

Further, a composition of the present invention comprises a tranquilizer which is a group of drugs that depresses the central nervous system causing relaxation, reduction of anxiety, drowsiness, and slowed breathing, respectivley. Different classes of tranquilizers are benzodiazepines such as valium, antihistamines, barbiturates, antidepressants, neuroleptics, and chlorhydrate from which one or more are selected to be comprised by the composition of the present invention. In some embodiments tranquilizers are expressed by plants which have sedative effects. Such plants are for example passion flower, valerian, hop, balm, St. John's wort, silverweed, peppermint, jasmine blossom, orange blossom, and lavender whereof one or more are selected for the present composition. The plant is used in the composition either in fresh or dry form. Alternatively an extract will be prepared from the plant, e.g., by soaking the plant, simmering it and/or preserving the plant and/or the extract isolated from the plant for example in an alcohol which is part of the composition according to an embodiment of the present invention. The plant is used in total or parts of it such as a leaf, blossom, stipe, paring, roots, corm and/or seed. The plant is harvested in the morning, during insolation or in the evening and night, respectively.

Moreover, the present composition comprises one or more tranquilizing components which are for example benzodiazepines such as valium, adinazolam, alprazolam, bentazepam, bretazenil, bromazepam, brotizolam, camazepam, chlordiazepoxide, cinazepam, cinolazepam, clobazam, clonazepam, clonazolam, delorazepam, diazepam, diclazepam, estazolam, ethyl carfluzepate, etizolam, ethyl loflazepate, flubromazepam, flunitrazepam, flurazepam, flutazolam, flutoprazepam, halazepam, ketazolam, loprazolam, lorazepam, lormetazepam, medazepam, mexazolam, midazolam, nifoxipam, nimetazepam, oxazepam, phenazepam, pinazepam, prazepam, premazepam, pyrazolam, quazepam, rilmazafone, temazepam, thienalprazolam, tetrazepam, triazolam, flumazenil, eszopiclone, zaleplon, zolpidem, zopiclone; antihistamines such as diphenhydramine, chlorpheniramine, loratadine; barbiturates such as methohexital, thiamyl, thiopental, amobarbital, pentobarbital, secobarbital, butalbital, butabarbita, phenobarbital, mephobarbital; antidepressants such as amitriptyline, clomipramine, desipramine, doxepin, imipramine, nortriptyline, protriptyline, trimipramine, citalopram, escitalopram, fluoxetine, fluvoxamine, paroxetine, sertraline; neuroleptics such as chlorpromazine, fluphenazine, haloperidol, molindone, thiothixene, thioridazine, trifluoperazine, loxapine, perphenazine, prochlorperazine, pimozide; and chlorhydrate.

The composition further comprises one or more halogens or halides for example brome, bromide, chlorine, chloride, iodine, iodide, fluorine and/or fluoride, wherein the halides are for example metal halides, organohalides, silver halide, hydrogen halide etc. In one embodiment, a halide is a colloidal halide which is for example incubated with an alcohol for e.g., 1 to 24 h, 1 to 12 h or 1 to 6 h, then the alcohol is decanted and used in a composition according to the present invention. In an embodiment the alcohol is ethanol. In an embodiment halogens or halides are prepared based on homeopathic methods and added to the composition of the present invention. For example 1 part halogen or halide is mixed with 9 parts of one or more excipients (e.g. as described in the present application) and the mixture is intensively shaken (e.g., 10 times), 1 part thereof is taken from the mixture and further mixed with 9 parts of one or more excipients (e.g. as described in the present application) and the mixture is intensively shaken (e.g., 10 times) etc. until the desired dilution is reached which is for example 1:1 million. Such preparation method is not limited to halogens and halides but can be used for any component of the present composition as well as for the composition before use in treating and/or preventing a condition, disorder or disease.

In addition, the present composition comprises one or more of a carotinoid. Carotenoids are generally split into two classes, xanthophyll (containing oxygen) such as lutein, astaxanthin, and/or zeaxanthin, and carotenes (purely hydrocarbons without oxygen) such as beta-carotene, alpha-carotene, and/or gamma-carotene. Carotenoids of the present invention are for example hydrocarbons such as lycopersene 7,8,11,12,15,7′,8′,11′,12′,15′-decahydro-γ,γ-carotene, phytofluene, hexahydrolycopene 15-cis-7,8,11,12,7′,8′-hexahydro-γ,γ-carotene, torulene 3′,4′-didehydro-β,γ-carotene, α-Zeacarotene 7′,8′-dihydro-ε,γ-carotene; alcohols such as alloxanthin, cynthiaxanthin, pectenoxanthin, cryptomonaxanthin (3R,3′R)-7,8,7′,8′-tetradehydro-β,β-carotene-3,3′-diol, crustaxanthin β,-carotene-3,4,3′,4′-tetrol, gazaniaxanthin (3R)-5′-cis-β,γ-caroten-3-ol, OH-chlorobactene 1′, 2′-dihydro-f,γ-caroten-1′-ol, loroxanthin β,ε-carotene-3,19,3′-triol, lutein (3R,3′R,6′R)-β,ε-carotene-3,3′-diol, lycoxanthin γ,γ-caroten-16-ol, rhodopin 1,2-dihydro-γ,γ-caroten-1-ol, rhodopinol aka warmingol 13-cis-1,2-dihydro-γ,γ-carotene-1,20-diol, saproxanthin 3′,4′-didehydro-1′,2′-dihydro-β,γ-carotene-3,1′-diol, zeaxanthin; glycosides such as oscillaxanthin 2,2′-bis(β-L-rhamnopyranosyloxy)-3,4,3′,4′-tetradehydro-1,2,1′,2′-tetrahydro-γ,γ-carotene-1,1′-diol, phleixanthophyll 1′-(β-D-glucopyranosyloxy)-3′,4′-didehydro-1′,2′-dihydro-β,γ-caroten-2′-ol; ethers such as rhodovibrin 1′-methoxy-3′,4′-didehydro-1,2,1′,2′-tetrahydro-γ,γ-caroten-1-ol, spheroidene 1-methoxy-3,4-didehydro-1,2,7′,8′-tetrahydro-γ,γ-carotene; epoxides such as diadinoxanthin 5,6-epoxy-7′,8′-didehydro-5,6-dihydro-carotene-3,3-diol, luteoxanthin 5,6: 5′,8′-diepoxy-5,6,5′,8′-tetrahydro-6,6-carotene-3,3′-diol, mutatoxanthin, citroxanthin, zeaxanthin furanoxide 5,8-epoxy-5,8-dihydro-β,β-carotene-3,3′-diol, neochrome 5′,8′-epoxy-6,7-didehydro-5,6,5′,8′-tetrahydro-6,6-carotene-3,5,3′-triol, foliachrome, trollichrome, vaucheriaxanthin 5′,6′-epoxy-6,7-didehydro-5,6,5′,6′-tetrahydro-β,β-carotene-3,5,19,3′-tetrol; aldehydes such as rhodopinal, warmingone 13-cis-1-hydroxy-1,2-dihydro-γ,γ-caroten-20-al, torularhodinaldehyde 3′,4′-didehydro-β,γ-caroten-16′-al; Acids and acid esters such as torularhodin 3′,4′-didehydro-β,γ-caroten-16′-oic acid, torularhodin methyl ester methyl 3′,4′-didehydro-β,γ-caroten-16′-oate; ketones such as astacene, astaxanthin, canthaxanthin aka aphanicin, chlorellaxanthin β,β-carotene-4,4′-dione, capsanthin (3R,3′S,5′R)-3,3′-dihydroxy-β,κ-caroten-6′-one, capsorubin (3S,5R,3′S,5′R)-3,3′-dihydroxy-κ,κ-carotene-6,6′-dione, cryptocapsin (3′R,5′R)-3′-hydroxy-β,κ-caroten-6′-one, 2,2′-diketospirilloxanthin 1,1′-dimethoxy-3,4,3′,4′-tetradehydro-1,2,1′,2′-tetrahydro-γ,γ-carotene-2,2′-dione, echinenone 6,6-caroten-4-one, 3′-hydroxyechinenone, flexixanthin 3,1′-dihydroxy-3′,4′-didehydro-1′,2′-dihydro-β,γ-caroten-4-one, 3-OH-canthaxanthin aka adonirubin aka phoenicoxanthin 3-hydroxy-β,β-carotene-4,4′-dione, hydroxyspheriodenone 1′-hydroxy-1-methoxy-3,4-didehydro-1,2,1′,2′,7′,8′-hexahydro-γ,γ-caroten-2-one, okenone 1′-methoxy-1′,2′-dihydro-c,γ-caroten-4′-one, pectenolone 3,3′-dihydroxy-7′,8′-didehydro-β,β-caroten-4-one, phoeniconone aka dehydroadonirubin 3-hydroxy-2,3-didehydro-6,6-carotene-4,4′-dione, phoenicopterone β,ε-caroten-4-one, rubixanthone 3-hydroxy-β,γ-caroten-4′-one, siphonaxanthin 3,19,3′-trihydroxy-7,8-dihydro-β,ε-caroten-8-one; esters of alcohols such as astacein 3,3′-bispalmitoyloxy-2,3,2′,3′-tetradehydro-β,β-carotene-4,4′-dione or 3,3′-dihydroxy-2,3,2′,3′-tetradehydro-β,β-carotene-4,4′-dione dipalmitate, fucoxanthin 3′-acetoxy-5,6-epoxy-3,5′-dihydroxy-6′,7′-didehydro-5,6,7,8,5′,6′-hexahydro-β,β-caroten-8-one, isofucoxanthin 3′-acetoxy-3,5,5′-trihydroxy-6′,7′-didehydro-5,8,5′,6′-tetrahydro-β,β-caroten-8-one, physalien, zeaxanthin (3R,3′R)-3,3′-bispalmitoyloxy-β,β-carotene or (3R,3′R)-β,β-carotene-3,3′-diol, siphonein 3,3′-dihydroxy-19-lauroyloxy-7,8-dihydro-β,ε-caroten-8-one or 3,19,3′-trihydroxy-7,8-dihydro-β,ε-caroten-8-one 19-laurate; apocarotenoids such as β-apo-2′-carotenal 3′,4′-didehydro-2′-apo-b-caroten-2′-al, apo-2-lycopenal, apo-6′-lycopenal 6′-apo-y-caroten-6′-al, azafrinaldehyde 5,6-dihydroxy-5,6-dihydro-10′-apo-β-caroten-10′-al, bixin 6′-methyl hydrogen 9′-cis-6,6′-diapocarotene-6,6′-dioate, citranaxanthin 5′,6′-dihydro-5′-apo-β-caroten-6′-one or 5′,6′-dihydro-5′-apo-18′-nor-6-caroten-6′-one or 6′-methyl-6′-apo-6-caroten-6′-one, crocetin 8,8′-diapo-8,8′-carotenedioic acid, crocetinsemialdehyde 8′-oxo-8,8′-diapo-8-carotenoic acid, crocin digentiobiosyl 8,8′-diapo-8,8′-carotenedioate, hopkinsiaxanthin 3-hydroxy-7,8-didehydro-7′,8′-dihydro-7′-apo-b-carotene-4,8′-dione or 3-hydroxy-8′-methyl-7,8-didehydro-8′-apo-b-carotene-4,8′-dione, methyl apo-6′-lycopenoate methyl 6′-apo-y-caroten-6′-oate, paracentrone 3,5-dihydroxy-6,7-didehydro-5,6,7′,8′-tetrahydro-7′-apo-b-caroten-8′-one or 3,5-dihydroxy-8′-methyl-6,7-didehydro-5,6-dihydro-8′-apo-b-caroten-8′-one, sintaxanthin 7′,8′-dihydro-7′-apo-b-caroten-8′-one or 8′-methyl-8′-apo-b-caroten-8′-one; nor- or seco-carotenoids such as actinioerythrin 3,3′-bisacyloxy-2,2′-dinor-b,b-carotene-4,4′-dione, β-carotenone 5,6:5′,6′-diseco-b,b-carotene-5,6,5′,6′-tetrone, peridinin 3′-acetoxy-5,6-epoxy-3,5′-dihydroxy-6′,7′-didehydro-5,6,5′,6′-tetrahydro-12′,13′,20′-trinor-b,b-caroten-19,11-olide, pyrrhoxanthininol 5,6-epoxy-3,3′-dihydroxy-7′,8′-didehydro-5,6-dihydro-12′,13′,20′-trinor-b,b-caroten-19,11-olide, semi-α-carotenone 5,6-seco-b,e-carotene-5,6-dione, semi-β-carotenone 5,6-seco-b,b-carotene-5,6-dione or 5′,6′-seco-b,b-carotene-5′,6′-dione, triphasiaxanthin 3-hydroxysemi-b-carotenone 3′-hydroxy-5,6-seco-b,b-carotene-5,6-dione or 3-hydroxy-5′,6′-seco-b,b-carotene-5′,6′-dione; retro-carotenoids and retro-apo-carotenoids such as eschscholtzxanthin 4′,5′-didehydro-4,5′-retro-b,b-carotene-3,3′-diol, eschscholtzxanthone 3′-hydroxy-4′,5′-didehydro-4,5′-retro-b,b-caroten-3-one, rhodoxanthin 4′,5′-didehydro-4,5′-retro-b,b-carotene-3,3′-dione, tangeraxanthin 3-hydroxy-5′-methyl-4,5′-retro-5′-apo-b-caroten-5′-one or 3-hydroxy-4,5′-retro-5′-apo-b-caroten-5′-one; higher carotenoids such as nonaprenoxanthin 2-(4-hydroxy-3-methyl-2-butenyl)-7′,8′,11′,12′-tetrahydro-e,y-carotene, decaprenoxanthin 2,2′-bis(4-hydroxy-3-methyl-2-butenyl)-e,e-carotene, C.p. 450 2-[4-hydroxy-3-(hydroxymethyl)-2-butenyl]-2′-(3-methyl-2-butenyl)-b,b-carotene, C.p. 473 2′-(4-hydroxy-3-methyl-2-butenyl)-2-(3-methyl-2-butenyl)-3′,4′-didehydro-1′,2′-dihydro-b,y-caroten-1′-ol, bacterioruberin 2,2′-bis(3-hydroxy-3-methylbutyl)-3,4,3′,4′-tetradehydro-1,2,1′,2′-tetrahydro-y,y-carotene.

Another component of the present invention is one or more grains such as cereal grains for example bajra (pearl millet), finger millet, fonio, foxtail millet, Kodo millet, Japanese millet, Job's Tears, maize (corn), pearl millet, proso millet, sorghum; such as cool-season (C3) cereals for example barley, oats, rice, rye, teff, triticale, wheat, wild rice; such as pseudocereal grains for example amaranth (Amaranth family), buckwheat (Smartweed family), quinoa (Amaranth family, formerly classified as Goosefoot family); such as grain legumes or pulses for example chickpeas, common beans, common peas (garden peas), fava beans, lentils, lima beans, lupins, mung beans, peanuts, pigeon peas, runner beans, soybeans; such as oilseeds for example black mustard, India mustard, rapeseed (including canola). In one embodiment, the grain is selected from the group consisting of wheat, rye, barley, oat, rice, corn, sesame and millet. One or more of these grains optionally form(s) part of the present composition, wherein the composition comprises a grain such as rice hull in an amount of for example 1 to 100 g, 5 to 80 g, 10 to 50 g, 20 to 25 or 30 g, or rice and/or sesame in an amount of for example 1 to 50 g, 5 to 30 or 40 g, 10 to 20 or 25g, or 1 to 10 g. The ratio of grains in the present composition is for example 1:1, 1:2, 1:5 or 1:10.

A composition of the present invention further comprises a neutral gel for example as vehicle of basic material of the composition or of a component of the composition. A neutral gel is a hydrophilic gel (hydrogel) or a lipophilic gel (oleogel), wherein the hydrogel comprises for example 50 to 95% water, 60 to 90% water, 70 to 85% water, or 80 to 90% water. A hydrogel comprises a swelling agent such as cellulose, gelatin, pectin, agar-agar and/or a derivative thereof. Further gelling agents are for example xanthan gum, sodium alginate polycarbophil, polysaccharides, natural gums, acacia, tragacanth, starch, cellulose derivatives such as carboxy methyl cellulose, hydroxyl propyl methyl cellulose, hydroxypropyl methylcellulose (Methocel K4 M), methacrylate polymers, polyvinyl pyrrolidone, bentonite, alginic acid, carbomer, ethyl cellulose, guar gum, hydroxyl ethyl cellulose, hydroxyl propyl cellulose, hydroxyethyl methylcellulose, glyceryl behenate, algae extracts, gums, polysaccharides, polyethylene oxide, poloxamer, pectins, hydrolysed proteins, polymers comprising pendant carboxylic acid groups, or esters thereof, polymers comprising pendant anhydrides of dicarboxylic acid groups and block co-polymers based on ethylene oxide and/or propylene oxide and the like or mixtures thereof. Moreover, the neutral gel optionally comprises glycerol, propyl glycol and/or a preserving agent.

The composition of the present invention may further comprise an active agent such as a chemotherapeutic and/or a conventional pharmaceutically acceptable excipient such as a binding agent, a flavoring agent, a filler, a lubricant, a disintegrant, and/or a wetting agent.

For oral administration, the pharmaceutical composition of the invention can take the form for example as mentioned previously prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). The pharmaceutical composition can be administered with a physiologically acceptable carrier to a subject. The term “carrier” refers for example to a diluent, adjuvant, excipient, or vehicle with which the therapeutic is administered. Such pharmaceutical carriers can be sterile liquids, such as water and oils, including those of petroleum, animal, vegetable or synthetic origin, such as peanut oil, soybean oil, mineral oil, sesame oil and the like. Water is for example a carrier when the pharmaceutical composition is administered intravenously. Saline solutions and aqueous dextrose and glycerol solutions can also be employed as liquid carriers, particularly for injectable solutions. Suitable pharmaceutical excipients include starch, glucose, lactose, sucrose, gelatin, malt, flour, chalk, silica gel, sodium stearate, glycerol monostearate, talc, dried skim milk, glycerol, propylene, glycol, water, ethanol and the like. The composition, if desired, can also contain minor amounts of wetting or emulsifying agents, or pH buffering agents. The composition can also comprise or contain buffer salts, flavoring, coloring and sweetening agents.

For administration by inhalation, the composition of the invention is conveniently delivered for example in the form of an aerosol spray presentation from a pressurised pack or a nebulizer, with the use of a suitable propellant (e.g., dichlorodifluoromethane, trichlorofluoromethane, dichlorotetrafluoroethane, carbon dioxide or other suitable gas). In the case of a pressurised aerosol, the dosage unit can be determined for example by providing a valve to deliver a metered amount. Capsules and cartridges of, for example, gelatine, for use in an inhaler or insufflator can be formulated containing a powder mix of the pharmaceutical composition of the invention and a suitable powder base such as lactose or starch.

The composition of the present invention is for example administered orally, topically, subcutaneously, intravenously, intramuscularly, via inhalation, nasally, sublingually, and/or into the eye. The composition is for example used inwardly and/or outwardly in form of a tablet, caplet, film, capsule, pill, effervescent tablet, gel, spray, solution, suspension, powder, suppository, ointment, lotion, cream, paste, aerosol, foam, bath additive, wet pack or tansdermal patch, e.g., for retard release.

A composition of the present invention is for exampled used (in a method) for treating and/or preventing a condition, disorder or disease such as pain; metabolic diseases such as cystinosis, cystinuria, Fabry disease, galactosemia, Gaucher disease, Hartnup disease, homocystinuria, Hunter syndrome, Lesch-Nyhan syndrome, maple syrup urine disease, Maroteaux-Lamy syndrome, Morquio syndrome, Niemann-Pick disease, polyketonuria, Pompe disease, porphyria, Scheie syndrome, Tay-Sachs syndrome, tyrosinemia, von Gierke disease; fatigue; absence of appetite; cramps; immune diseases such as allergy, lupus, scleroderma, vasculitis, diabetes, graves disease, multiple sclerosis, rheumatoid arthritis; inflammatory disease such as Alzheimer disease, arthritis, asthma, Crohn's disease, colitis, dermatitis, hepatitis, bowel syndrome, nephritis, Parkinson's disease; cancer such as breast cancer, cerebral tumor, pancreas tumor, colon cancer, melanoma; neurological and/or psychiatric diseases. In an embodiment the composition is administered accompanying cancer treatment, it reduces pain, increases appetite, strengthens immune system and immune defense, strengthens body functions, detoxifies, accelerates cell renewal, and/or reduces fatigue.

The composition is used in treating and/or preventing a condition, disorder or disease in a mammalian such as a human or animal such as a domestic or pet animal for example a dog, cat, horse, donkey, cattle, sheep, pig, rabbit or goat; bird such as duck, chicken or goose; or fish.

The composition is administered for example in an acute phase of a disease or continuously, it is administered for example 1 to 5 times/day, 1 to 3 times/day or 1 time/day, 1 to 3 times/week or 1 to 3 times/month. The composition of the present invention is for example used as a medicament, e.g., a homeopathic medicament, or a dietary supplement. The composition of the invention described herein can be administered to the subject at a suitable dose.

In an embodiment of the present invention the composition comprises or consists of for example

TABLE 1

| | |
|---|---|
| Neutral gel | e.g., 50-300 g |
| Chrom II and/or III | e.g., 100-500 μg |
| Manganese | e.g., 10-50 mg |
| Lutein | e.g., 10-30 mg |
| Silicon, e.g., powder | e.g., 10-50 g |
| Brom | e.g., 1-10 drops |
| Rice hull e.g., powder | e.g., 10-50 g |
| Rice e.g., powder | e.g., 1-10 g |
| Glauber salt | e.g., 10-50 g |
| Extract of silverweed | e.g., 20-60 drops or 0.5-5 ml |

In an embodiment for a method of preparation of a composition of the present invention, all components of Table 1 are mixed and heated to about 15 to 40° C., 20 to 35° C., or 25 to 30° C. for 5 to 60 min, 10 to 50 min or 20 to 30 min.

### EXAMPLES

In the following examples are provided to better illustrated the present invention which is not bound to or limited by these examples.

### Example 1

A composition of the present invention consisting of

| | | |
|---|---|---|
| Neutral gel | 80 | g |
| Chrom II | 120 | μg |
| Manganese | 15 | mg |
| Lutein | 20 | mg |
| Silicon powder | 18 | g |
| Brom | 3 | drops |
| Rice hull powder | 23 | g |
| Rice powder and/or sesame (1:1) | 3 | g |
| Glauber salt | 25 | g |

| | |
|---|---|
| Extract of silverweed | 30 drops or 1.2 ml |

was administered to a 70 year old male patient having a very bad general condition, cell mutations, and feeling weakness as well as severe pain. The liquid composition was administered 3× per day orally in the amount of a teaspoon. After administration of the composition, the pain significantly decreased, appetite increased and the general condition improved.

### Example 2

A composition of the present invention comprising

| | | |
|---|---|---|
| Neutral gel | 250 | g |
| Chrom III | 180 | μg |
| Manganese | 20 | mg |
| Lutein | 15 | mg |
| Silicon powder | 38 | g |
| Brom | 5 | drops |
| Rice hull powder | 45 | g |
| Rice powder and/or sesame (1:1) | 8 | g |
| Glauber salt | 26 | g |

| | |
|---|---|
| Extract of silverweed | 40 drops or 2.3 ml |

was administered to a female patient in the age of 50 having a very bad general condition and low body weight after chemotherapy. After 10 days of treatment with the above mentioned composition in form of a tablet led to a general invigoration of the patient, severe reduction of pain and increase of body weight.

1. A composition comprising a metal and/or a semimetal, a sodium salt, a tranquilizer which is provided by a plant selected from the group consisting of passion flower, valerian, hop, balm, St. John's wort, silverweed, peppermint, jasmine blossom, orange blossom, and lavender, a halogen, a carotenoid, grain, and a neutral gel.

2. The composition according to claim 1, wherein the metal and/or semimetal is selected from the group consisting of chromium, manganese, silicon, iron, cobalt, copper, zinc, calcium, potassium, magnesium and sodium.

3. The composition according to claim 1, wherein the sodium salt is selected from the group consisting of NaCl, Na₂SO₄, Na₂CO₃, NaBr, Na₂CrO₄, NaHSO₄, NaOH, NaMnO₄, Na₃PO₄, Na₂HPO₄, NaH₂PO₄ and Glauber salt.

4. The composition according to claim 1, wherein the tranquilizer is selected from the group consisting of a benzodiazepine, an antihistamine, a barbiturate, an antidepressant, a neuroleptic and a chlorhydrate.

5. The composition according to claim 1, wherein the tranquilizer is selected from the group consisting of benzodiazepines such as valium, antihistamines, barbiturates, antidepressants, neuroleptics, and chlorhydrate.

6. The composition according to claim 1, wherein the halogen is bromide, chloride, iodide, and/or fluoride.

7. The composition according to claim 1, wherein the carotenoid is selected from the group consisting of carotene such as beta-carotene, alpha-carotene, gamma-carotene, beta-cryptoxanthin, lutein, astaxanthin, and zeaxanthin.

8. The composition according to claim 1, wherein the grain is selected from the group consisting of wheat, rye, barley, oat, rice, corn, sesame and millet.

9. The composition according to claim 1, wherein the neutral gel comprises agar-agar, cellulose, gelatin, pectin and/or a derivative thereof.

10. The composition according to claim 1, which is administered orally, topically, subcutaneously, intravenously, intramuscularly, via inhalation, nasally, sublingually, and/or into the eye.

11. The composition according to claim 1 for use in treating and/or preventing pain, metabolic diseases, fatigue, absence of appetite, cramps, immune diseases, inflammatory disease, cancer, neurological and/or psychiatric diseases.

12. The composition according to claim 1, wherein the composition is administered 1 to 3 times/day, 1 to 3 times/week or 1 to 3 times/month.

13. The composition according to claim 1 which is a medicament or a dietary supplement.

14. The composition according to claim 13 wherein the medicament is a homeopathic medicament.

15. A prophylactic or therapeutic method, the method comprising providing to an individual in need thereof a composition, the composition comprising a metal and/or a semimetal, a sodium salt, a tranquilizer from a plant selected from the group consisting of passion flower, valerian, hop, balm, St. John's wort, silverweed, peppermint, jasmine blossom, orange blossom, and lavender, a halogen, a carotenoid, grain, and a neutral gel, as therapy for or prevention of pain, metabolic diseases, fatigue, absence of appetite, cramps, immune diseases, inflammatory disease, cancer, neurological and/or psychiatric diseases.

16. The method of claim 15 where the composition is administered orally, topically, subcutaneously, intravenously, intramuscularly, via inhalation, nasally, sublingually, and/or into the eye.

17. The method of claim 15 where the composition is administered to the individual 1 to 3 times/day, 1 to 3 times/week, or 1 to 3 times/month.

18. The method of claim 15 where the composition administered is a medicament or a dietary supplement.

19. The method of claim 15 where the composition administered is a homeopathic medicament.
